(19) United States
(12) Patent Application Publication
(10) Pub. No.: US 2015/0343054 A1
(43) Pub. Date: Dec. 3, 2015

(54) Heroin Haptens, Immunoconjugates and Related Uses

(71) Applicant: Kim D. Janda, La Jolla, CA (US)

(72) Inventor: Kim D. Janda, La Jolla, CA (US)

(21) Appl. No.: 14/367,511

(22) PCT Filed: Dec. 21, 2011
(86) PCT No.: PCT/US11/01997

§ 371 Date: Jul. 16, 2015

(51) Int. Cl. A61K 39/385; C07K 16/44; A61K 47/48; C07D 489/02

(52) U.S. Cl. CPC A61K 39/385; C07D 489/02; C07K 16/44; A61K 47/4833; C07K 2317/92; A61K 2039/6081

(57) ABSTRACT

The present invention provides novel heroin hapten compounds and heroin immunoconjugates which can be used for in vivo production of antibodies that specifically bind to heroin and its psychoactive metabolites. The invention also provides methods of using vaccines comprising the heroin immunoconjugates in active or passive immunization protocols. The compositions and methods of the invention are useful for prevention and treatment of heroin addiction.

### STATEMENT OF GOVERNMENT SUPPORT

This invention was made with government support under grant number DA026625 awarded by the National Institutes of Health. The government has certain rights in the invention.

### BACKGROUND OF THE INVENTION

Injection drug abuse is a debilitating worldwide epidemic, comprised of an estimated 14 million global users. Of the commonly abused injection drugs, opiates can be considered as the primary source for abuse. When considering the spectrum of negative effects from opiate abuse, heroin is especially destructive. Additionally, heroin abuse and addiction can be viewed as a driving force in the spread of HIV, with an estimated 10% of all new HIV infections attributed to injection drug users. Thus, an effective therapy targeting the successful rehabilitation of opiate abusers represents an attractive goal to improve health throughout the population.

Treatment options for heroin addiction rehabilitation address both initial detoxification issues involved with heroin use cessation as well as assisting the addict in maintaining an abstinent lifestyle. However, these options suffer from serious side effects. For example, long lasting opioid agonists including methadone, levo-methadyl acetate and buprenorphine are used to prevent the negative consequences of withdrawal. Heroin replacement therapy with agonistic compounds still exposes the patient to opiates, and the subject remains dependent and vulnerable to relapse. In addition, opiate replacement therapies are often unavailable to addicts, particularly in developing countries, due to lack of infrastructure to maintain a reliable supply or denial of replacement access altogether. Another treatment approach employing opioid antagonistic compounds such as naloxone or naltrexone blocks the body's endogenous opioids (endomorphins, enkephalins), potentially resulting in dysphoric symptoms for the patient, and as a result compliance is an issue.

There is a need in the art for better means for treating heroin addiction. The present invention addresses this and other unfulfilled needs in the art.

### SUMMARY OF THE INVENTION

In one aspect, the invention provides a hapten compound of formula (I):

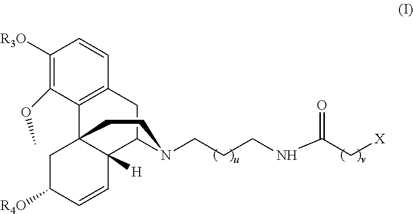

wherein R₃ and R₄ are each —H or an acyl group, u and v are each an integer from about 0 to about 6, and X is a linker moiety. The linker moiety can be selected from the group consisting of:
—Y,
—CH₃,
—(CH₂)ₙCOY,
—(CH₂)ₙCNY,
—(CH₂)ₙY,
—CH═Y,
—CH(O)CH₂,
—CH(OH)CH₂Y,
—(CH₂)ₙCH(OH)CH₂Y,
—(CH₂)ₙCH(O)CH₂Y,
—C₆H₅,
—(CH₂)ₙY
—NH(CH₂)ₙY,
—CH₂OCO(CH2)ₙCOY,
—CH₂OCO(CH₂)ₙCNY,
—CH₂Z(CH₂)ₙY,
—(CH₂)n-C₆H₁₀—(CH₂)ₘ—COY,
—(CH₂)n-C₆H₄—(CH₂)ₘ—COY,
—NH(CH₂)ₙCOY,
—NH(CH₂)ₖC₆H₁₀—(CH₂)ₘCOY,
—NH(CH₂)ₘC₆H₄—(CH₂)ₚCOY,
—NHCO(CH₂)ₙCOY,
—NHCO(CH₂)ₖC₆H₁₀—(CH₂)ₘCOY,
—NHCO(CH₂)ₘC₆H₄—(CH₂)ₚCOY,
—C≡C—(CH₂)ₙNHY,
—C≡C—(CH₂)ₙCOY,
—C≡C—(CH₂)ₙC₆H₁₀—(CH₂)ₘCOY,
—C≡C—(CH₂)ₙC₆H₁₀—(CH₂)ₘNHY,
—C≡C—(CH₂)ₘC₆H₄—(CH₂)ₚCOY,
—C≡C—(CH₂)ₘC₆H₄—(CH₂)ₚNHY,
—CH═CH—(CH₂)ₙNHY,
—CH═CH—(CH₂)ₙCOY,
—CH═CH—(CH₂)ₙC₆H₁₀—(CH₂)ₘCOY,
—CH═CH—(CH₂)ₙC₆H₁₀—(CH₂)ₘNHY,
—CH═CH—CH₂)ₘC₆H₄—(CH₂)ₚCOY,
—CH═CH—(CH₂)ₘC₆H₄—(CH₂)ₚNHY,
—(CH₂)ₙ—R3-(CH₂)ᵣ—R₂—Y,
—Z(CH₂)ₙY,
—ZCO(CH₂)ₙCOY
   wherein n is an integer from about 1 to about 20; m is an integer from about 0 to about 6; k is an integer from about 0 to about 20; p is an integer from about 0 to about 6; r is an integer from about 1 to about 20; Z is selected from the group consisting of —O—, —CH₂—, and —NH—; R₁ and R₂ are independently selected from the group consisting of —NHCO—, —CONH—, —CONHNH—, —NHNHCO—, —NHCONH—, —CONHNHCO—, and —S—S—; and Y is selected from the group consisting of —SH, —H, —OH, ═CH₂, —CH₃, —OCH₃, —COOH, halogen, acyl, 2-nitro-4-sulfobenzoate, N-oxysuccinimididate, N-maleimides, imino acylate, isocyanates, isothiocyanates, haloformate, vinylsulfone, imidoester, phenylglyoxalate, hydrazide, azido, amino, and N-hydroxysuccinimidate.

In some haptens of formula (I), R₃ and R₄ are each —H. In some other haptens, the R₃ and R₄ groups are both —COCH₃. In some haptens of formula (I), u and/or v are 2. In some preferred embodiments, the linker moiety X in the haptens is —SH. In some of these haptens, R₃ and R₄ are both —H, u and v are both 2, and X is —SH. In some other preferred embodiments, R₃ and R₄ are both —COCH₃, u and v are both 2, and X is —SH.

In a related aspect, the invention provides an immunoconjugate of formula (II):

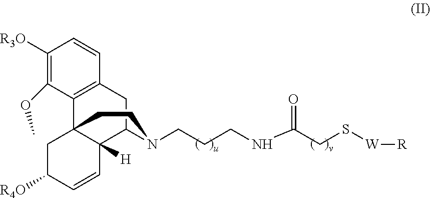

wherein R₃ and R₄ are each —H or an acyl group, u and v are each an integer from about 0 to about 6, and W is a linker moiety that is covalently linked to a carrier moiety R. In some embodiments, the linker moiety W has a structure shown in formula (III):

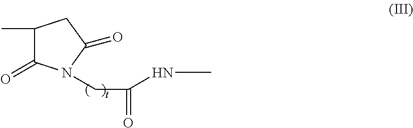

wherein t is an integer from about 1 to about 10. In some immunoconjugates of the invention, the R₃ and R₄ groups are each —H. In some other immunoconjugates, the R₃ and R₄ groups are each —COCH₃.

Various carrier moieties can be used in the immunoconjugates of the invention. For example, the carrier moiety R can be selected from the group comprising keyhole limpet hemocyanin (KLH), edestin, thyroglobulin, human serum albumin, sheep red blood cells (sheep erythrocytes), tetanus toxoid (TT), diphtheria toxoid, cholera toxoid, polyamino acids, D-lysine, D-glutamic acid, members of the LTB family of bacterial toxins, retrovirus nucleoprotein (retro NP), rabies ribonucleoprotein (rabies RNP), vesicular stomatitis virus nucleocapsid protein (VSV-N), recombinant pox virus subunits, and bovine serum albumin (BSA). In some preferred embodiments, the carrier moiety is KLH or BSA. In some of these immunoconjugates, the R₃ and R₄ groups are each —H, u and v are each 2, and t is 3. In some other preferred embodiments, the R₃ and R₄ groups are each —COCH₃, u and v are each 2, t is 3, and the carrier moiety is KLH or BSA. In a related aspect, the invention provides compositions which contain an immunologically effective amount of an immunoconjugate disclosed herein and a physiologically acceptable vehicle. The compositions can optionally further contain an adjuvant.

In another aspect, the invention provides methods of inducing an anti-heroin immune response in a subject. The methods involve immunizing the subject with an immunologically effective amount of a therapeutic composition that comprises a heroin immunoconjugate described herein. In some of these methods, the heroin immunoconjugate has a structure shown in formula (II), wherein R₃ and R₄ are each —COCH₃, u and v are each 2, the carrier moiety is KLH, and W is

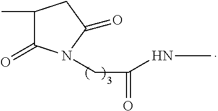

In another aspect, the invention provides isolated or substantially purified antibodies that bind to the heroin immunoconjugates described herein. Some of the antibodies can specifically bind to heroin and one or more of its psychoactive metabolites, acetylmorphine (6AM), morphine-6-Glucuronide (M6G) and morphine. In some preferred embodiments, the antibodies bind to heroin or acetylmorphine (6AM) with a dissociation constant of about 10 nM to about 20 μM.

A further understanding of the nature and advantages of the present invention may be realized by reference to the remaining portions of the specification and claims.

### BRIEF DESCRIPTION OF THE DRAWINGS

FIG. 1 shows structures of heroin and its psychoactive metabolites, 6-acetylmorphine (6AM), morphine, and morphine-6-glucuronide (M6G).

FIG. 2 shows the scheme of synthesis of heroin haptens 1-2 and generation of appropriate hapten-protein conjugates.

FIG. 3 shows titer levels of antibodies generated by heroin vaccines over the course of 165 days. Vertical arrows represent booster injections at t=14, 28, 53, 108 and 151 days. Data represented are the pooled sera mean value ±SEM.

FIGS. 4A-4D show that heroin-like vaccine selectively blocks the thermal and mechanical antinociceptive effects of heroin in rats. (A and B) Systemic injection of heroin (1 mg/kg, s.c.) produced robust decreases in both thermal (A) nociceptive sensitivity as measured by hot plate, and mechanical sensitivity (B) as measured by von Frey filament testing. This antinociceptive effect of heroin was fully reversed in rats which received the heroin-like vaccine. The morphine vaccine blunted the thermal nociceptive effect of heroin if compared to the control. But the thermal nociceptive effect of heroin in rats receiving the morphine vaccine was still significantly elevated from baseline. The morphine-like vaccine also did not alter mechanical sensitivity. (C and D) Injection of the structurally similar opiate oxycodone produced similar thermal and mechanical insensitivity as seen with heroin. Neither the heroin-like nor morphine-like vaccine altered antinociceptive responses to oxycodone. N=7-8 per group, ***p<0.001, 30 min post-drug versus baseline; #p<0.05, ###p<0.001, versus KLH response post-drug.

FIGS. 5A-5D show that heroin vaccine blocks acquisition of heroin self-administration by rats. (A and B) Acquisition of heroin self-administration is prevented in a subset of rats vaccinated against heroin, but not morphine. The percentage of animals that obtain the acquisition criteria, maintaining at least 3 consecutive sessions of 3 infusions (60 μg/kg/infusion) or more, is significantly lower in a group of rats receiving heroin vaccination compared to KLH controls (p<0.05). Only 3 of 7 rats receiving heroin vaccination achieved criteria. Conversely, the vaccine designed to be selective against morphine did not alter acquisition of heroin self-administration (p=0.96). (C and D) Vaccines targeted against heroin or morphine do not alter the acquisition of self-administration of a natural reward. Animals were trained to press on an active for 0.1 ml of sweetened solution containing 0.125% w/v saccharine and 3% w/v glucose. When examining the percentage of animals that acquired the response criteria for the sweetened solution, defined as consecutive days of at least 30 or more responses, survival analysis revealed no significant differences based on vaccination treatment (Heroin: p=0.63, Morphine: p=0.22 vs KLH control). N=7-8 per group.

FIG. 6 illustrates heroin metabolic pathway and the immunochemically dynamic nature of the heroin vaccines of the invention.

### DETAILED DESCRIPTION OF THE INVENTION

### I. Overview

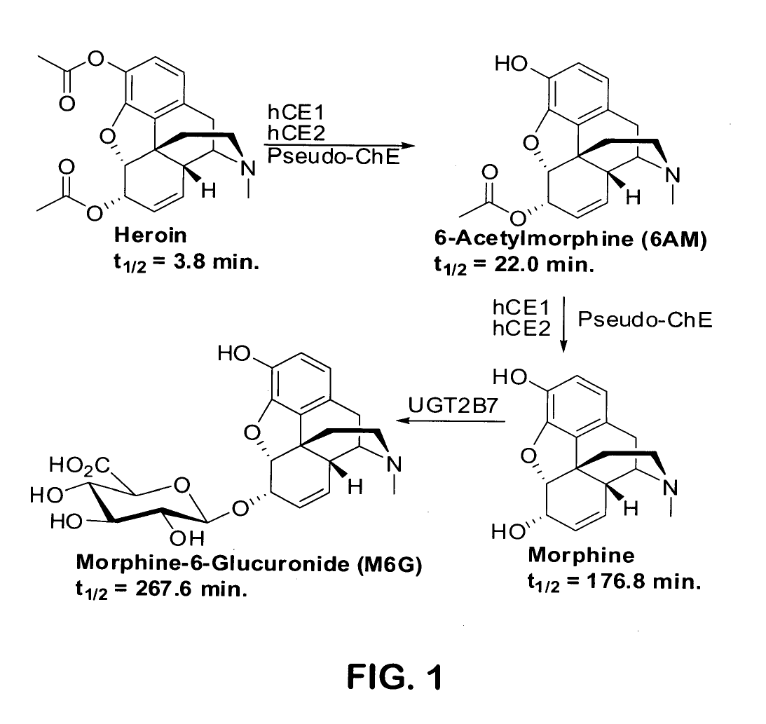

The present invention is predicated in part on a new class of heroin haptens and hapten-carrier immunoconjugates conceived by the present inventor which are useful in immunopharmacotherapy for the treatment of heroin addiction. The major challenge in the construction of an effective hapten-protein conjugate for a heroin vaccine stems from the inherent susceptibility of heroin to be enzymatically degraded into the psychoactive metabolites 6-acetylmorphine (6AM) and morphine. An additional psychoactive metabolite, morphine-6-glucuronide (M6G), stemming from phase II metabolism, is also formed in humans, but not rodents (FIG. 1). Critical from an immunopharmacodynamic standpoint is that while heroin and its psychoactive metabolites are structurally similar, they vary in lipophilicity, and thus the ability to cross the blood brain barrier (BBB). As such, the lipophilic molecules heroin and 6AM readily cross the BBB, while the less lipophilic morphine traverses the BBB slowly. Additionally, once heroin and 6AM cross the BBB, they are rapidly hydrolyzed to morphine, and can be considered as sequestered within the brain. Thus, instead of a broad immune response to the opiate scaffolding with equal affinity, a more successful vaccine candidate should preferentially bind the major lipophilic serum psychoactive components before crossing the BBB and gaining access to opioid receptors in the brain.

The present inventor generated novel heroin haptens and immunoconjugates for eliciting immune responses against heroin and its psychoactive metabolites. Since antibodies are unable to cross the BBB, the heroin vaccines of the invention were designed to be as efficacious as possible by eliciting a multi-drug immune response with 6AM and heroin being the primary targets in order to block the passage of these lipophilic psychoactive molecules into the brain. Unlike hapten designs reported in the art (e.g., Bonese et al., Nature 252: 708-10, 1974; and Anton et al., Vaccine 24: 3232-3240, 2006), the inventor employed an alkyl linker attached at the bridgehead nitrogen, instead of an ester at the 6′-position, for attachment of heroin haptens to carrier proteins. The haptens of the invention allowed the display of crucial structural modality found within the heroin scaffolding such that both immune recognition as well as possible novel adjuvant effects are accessed. Also, the immunoconjugates of the invention used a modular linker to allow for facile comparison between different haptens.

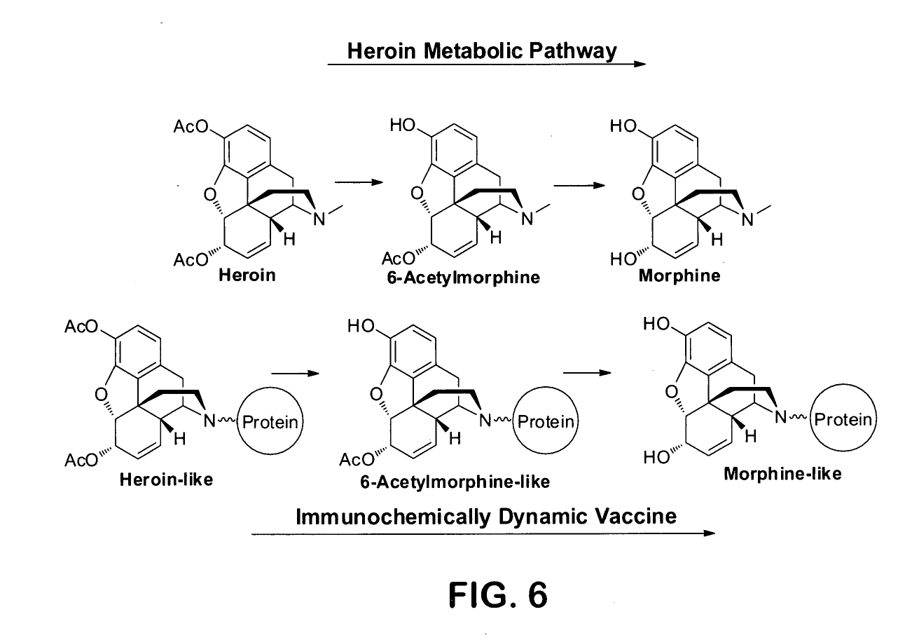

Additionally, the heroin haptens and immunoconjugates of the invention are designed to take advantage of the slow release from the shielded environment of Alum. See, e.g., Glenny et al., J. Pathol. Bacteriol. 1926, 29, 31-40; and Marrack et al., Nat. Rev. 2009, 9, 287-293. The vaccines can thus minimize the rapid enzymatic heroin ester hydrolysis and enhance heroin's structural integrity over its serum lability at physiological pH and temperature. Due to slow adjuvant desorption, the vaccines would provide a steady and chemically dynamic source of multiple drug-like antigens for presentation to the immune system (FIG. 6). As a result, the vaccines can generate a high titer immune response to 6AM, and to a lesser extent heroin, which in turn prevents acquisition of heroin self-administration and other centrally-mediated heroin effects in rodents. On the other hand, the morphine-like immunoconjugates, which also produces a high titer immune response, but primarily to morphine, are not effective for prevention of heroin administration acquisition due to its inability to peripherally bind 6AM. As exemplifications, heroin-like immunoconjugate 11b, while singular in its starting point, could challenge the immune system with multiple hapten-like chemical epitopes including heroin, 6AM and morphine, leading to a honed heterologous antibody response to these three opioids. On the other hand, morphine-like immunoconjugates such as immunoconjugate 12b are ‘true’ singular haptens without labile esters and would elicit antibodies highly specific to morphine.

Effective vaccines should also be able to elicit a rapid, high titer immune response from a minimum number of inoculations. As exemplified herein, the vaccines of the invention (e.g., vaccines with immunoconjugates 11b and 12b) generated high titers of antibodies (e.g., about :80,000 and 1:149,000, respectively, for immunoconjugates 11b and 12b) after only 28 days and two injections (1 boost). In addition, the maximal antibody titers from haptens 11b and 12b were ≈1:122,000 and 1:160,000, respectively, and these levels were reached after only three injections (2 boosts).

The vaccines of the invention are able to generate not only a high titer immune response, but also polyclonal antibodies capable of differentiating between extremely similar opiates. As determined by competition ELISA, the heroin vaccines of the invention (e.g., a vaccine based on heroin-like immunoconjugate-11b) can generate antibodies with outstanding affinity for 6AM, and excellent binding for heroin/morphine. Conversely, morphine-like immunoconjugate-12b generates antibodies with high affinity for morphine, but reduced binding for heroin and no affinity for 6AM. In addition, neither of these sets of polyclonal antibodies have any appreciable affinity for the structurally similar opioids morphine-6-glucuronide, codeine, naltrexone, oxycodone and naloxone, the opioid peptides endomorphin-2 and Leu-enkephalin, and the structurally dissimilar opioid agonist methadone. As a confirmation of the antibody specificities, rats vaccinated with the heroin-like or morphine-like immunoconjugates displayed significant antinociception after injection of oxycodone (i.e., the vaccines have not effect against oxycodone). This result is particularly gratifying given oxycodone's structural similarity to heroin and morphine.

Accordingly, the invention provides novel heroin hapten compounds, and immunoconjugates or vaccines comprising the haptens. The invention also provides therapeutic methods of using the immunoconjugates to treat subjects with heroin dependence or addiction. The immunoconjugates and vaccines of the invention could minimize the reinforcing effects of heroin, and provide a potential, highly useful, additional treatment option. Antibodies generated by the vaccines are specific only for heroin and its psychoactive metabolites. They can act as an opiate antagonist without the negative side effects in other therapies reported in the art. They are also suitable for use in combination with synthetic opioid replacement therapy.

The following sections provide more detailed guidance for practicing the present invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

As used herein, the term “adjuvant” refers to immunological agents that may stimulate the immune system of a subject and increase the response to a vaccine, without having any specific antigenic effect in itself. It encompasses any substance that acts to accelerate, prolong, or enhance antigen-specific immune responses when used in combination with specific vaccine antigens. Thus, an adjuvant suitable for the present invention is capable of enhancing the immune response against the immunoconjugates described herein.

As used herein, the term “hapten” refers to a small molecule which elicits a detectable immune response when attached to a carrier moiety. When constructed as an immunoconjugate, the hapten is characterized as the specificity-determining portion of the immunoconjugate.

“Active immunization” refers to the induction of an immune response in a subject by providing an antigen, for example, an immunoconjugate. Immunoconjugates are suitably included in a pharmaceutical composition containing a physiologically acceptable vehicle or carrier such that an immunologically effective amount of the immunoconjugate can be delivered to a subject.

A “carrier moiety,” as used herein, refers to a conjugation partner capable of enhancing the immunogenicity of the hapten. Carrier moieties are well known in the art and are generally proteins.

Heroin (diacetylmorphine), also known as diamorphine (BAN), diacetylmorphine or morphine diacetate, is an opioid analgesic synthesized from morphine, a derivative of the opium poppy. It is the 3,6-diacetyl ester of morphine, and functions as a morphine prodrug (meaning that it is metabolically converted to morphine inside the body). The white crystalline form considered “pure heroin” is usually the hydrochloride salt, diacetylmorphine hydrochloride. As with other opioids, diacetylmorphine is used as both an analgesic and a recreational drug. Frequent and regular administration is associated with tolerance and physical dependence, which may develop into addiction. Internationally, diacetylmorphine is controlled under Schedules I and IV of the Single Convention on Narcotic Drugs. It is illegal to manufacture, possess, or sell diacetylmorphine without a license in almost every country.

An “immunologically effective amount” means an amount of an immunogen (e.g., heroin or a heroin immunoconjugate disclosed herein) which is capable of inducing an immune response against the immunogen and/or generating antibodies specific for the immunogen or other agents which share immunological features of the immunogen of interest, e.g., heroin or its metabolite 6AM.

“Passive immunization” refers to short-term immunization achieved by the transfer of antibodies to a subject.

A “physiologically acceptable” vehicle is any vehicle or carrier that is suitable for in vivo administration (e.g., oral, transdermal, intramuscular, or parenteral administration) or in vitro use, i.e. cell culture.

The term “subject” refers to a vertebrate, suitably a mammal, more suitably a human.

Vaccine refers to a biological preparation that, when administered to a subject, elicits an immune response (including production of specific antibodies) against an agent (e.g., heroin) or that improves immunity to a particular disease. A vaccine typically contains a small amount of an immunogen (e.g., a heroin-like compound, derivative or metabolite) that immunologically resembles the agent of interest or a microorganism. The immunogen stimulates the body's immune system to recognize the agent as foreign, destroy it, and “remember” it, so that the immune system can more easily recognize and destroy the agent that it later encounters.

### III. Design and Synthesis of Heroin Haptens and Immunoconjugates

Because heroin is inherently non-immunogenic, the inventors have designed compounds that have structural and stereochemical features of heroin such that antibodies to these compounds will cross-react with heroin. Haptens in accordance with the present invention may be synthesized de novo or from a heroin-related compound. In some embodiments, heroin or a heroin derivative compound is employed as the starting material in synthesis of the haptens. In other embodiments, the heroin haptens can be generated by de novo synthesis in accordance with standard chemical methods well known in the art. The haptens of the present invention can be coupled with a carrier protein so that they can elicit an enhanced immune response in a subject. The immune response includes the production of hapten-specific antibodies which can cross-react with heroin and/or psychoactive metabolites of heroin (e.g., 6AM).

Some haptens in accordance with the invention have the structure shown in formula (I):

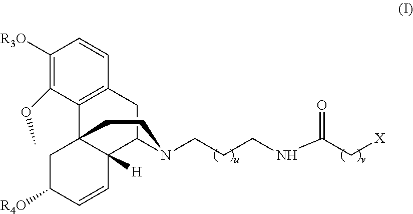

In formula (I), R₃ and R₄ can be any group that maintains the immunogenicity of heroin and/or morphine. In preferred embodiments, R₃ and R₄ are each —H or an acyl group. u and v in formula (I) are each an integer, e.g., from about 0 to about 6. X in formula (I) is a linker moiety. The linker moiety X can be any chemical group that is able to conjugate a carrier protein to the hapten at the bridgehead nitrogen in the hapten scaffold. Typically, the linker moiety is capable of reacting with an activated carrier protein to form a covalent linkage. Various linker moieties suitable for the haptens of the invention are described herein. In some exemplified embodiments, the linker moiety group is an —SH group.

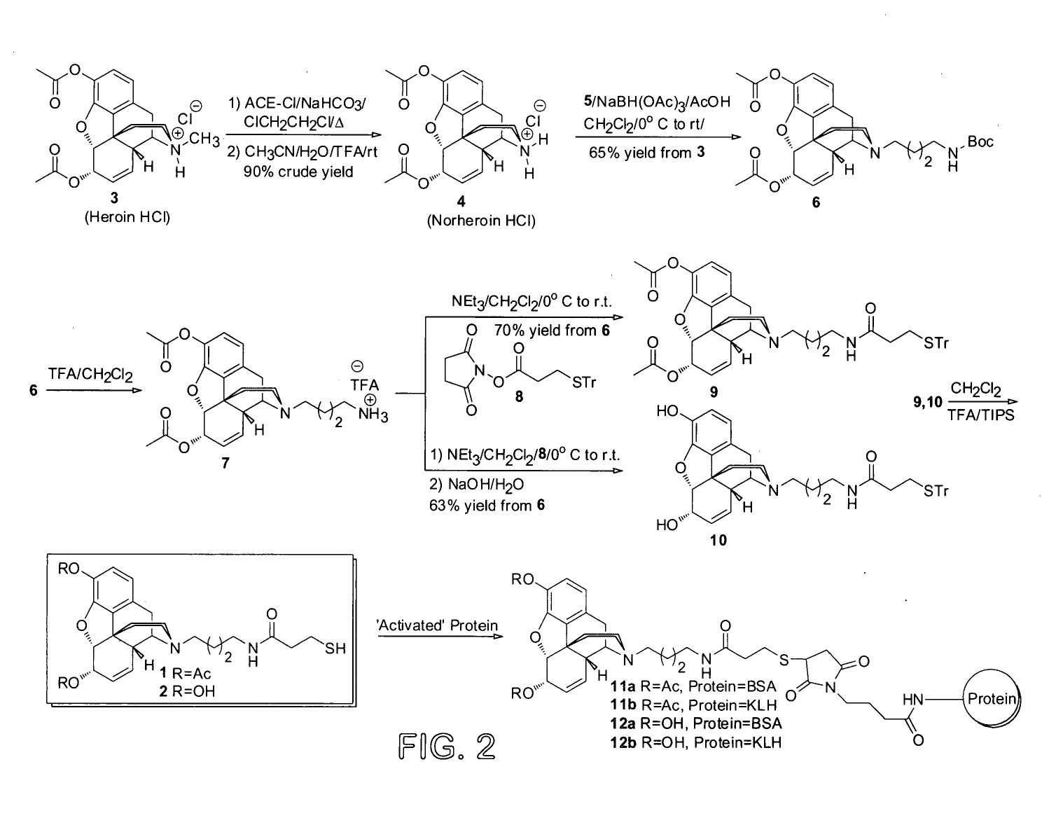

Some haptens of the invention are intended for generating antibody responses primarily against heroin and heroin metabolites other than morphine. In these haptens, the R₃ and R₄ groups are each —COCH₃. These haptens may be more specifically termed heroin-like haptens. Some other haptens of the invention are intended for generating antibody response primarily against morphine. In these haptens, the R₃ and R₄ groups are each —H. These haptens may also be more narrowly termed morphine-like haptens. In some preferred embodiments, u and v found within the amine and amide groups respectively connecting the bridgehead nitrogen and the linker moiety X are each 2. In some exemplified embodiments, the linker moiety X in the heroin-like or morphine-like haptens comprises an SH group (see, e.g., haptens 1-2 in FIG. 2).

Haptens of the invention may be synthetically derived to mimic the molecular features of heroin and its psychoactive metabolites. The hapten may be synthesized with or without the use of heroin or heroin derivatives as a reactant in the synthesis process. Exemplary methods of producing the hapten of formula (I) are described in the Examples below. An important aspect of the hapten structure of the invention is the use of the bridgehead nitrogen as linker attachment point, not the 6′-position on morphine/heroin scaffold as used in other heroin hapten designs. As demonstrated in the Examples herein, the novel design of the heroin haptens and resulting immunoconjugates of the haptens and carrier proteins, as well as the use of Alum adjuvant, allows generation of a heterologous high titer immune response against heroin and its psychoactive metabolites, 6AM and morphine. Examples of the heroin haptens of the present invention are shown in FIG. 2.

The haptens of the invention as described above can be linked to a carrier moiety to generate heroin immunoconjugates (e.g., heroin-like or morphine-like immunoconjugates). The immunoconjugates can be readily produced using standard methods known in the art. To generate the immunoconjugates, the heroin hapten can be covalently or non-covalently conjugated to the carrier moiety. Typically, the linker moiety X is conjugated to the carrier moiety via a covalent bond. Depending on the functional group in the linker moiety X and the carrier moiety, various covalent bonds can be used to conjugate the heroin hapten to the carrier moiety. In some embodiments, the linker moiety is first activated to generate a functional group that can readily react with an amino acid residue in the carrier moiety to form a covalent linkage. In some other embodiments, the carrier moiety can be modified with a derivatizing molecule or spacer molecule in order to generate a functional group for reacting with the heroin hapten. Derivatizing molecules suitable for practicing the present invention are well-known in the art. In some preferred embodiments, the heroin hapten is conjugated to an activated carrier moiety via a —SH linker moiety to form a thioether bond. Specific examples of immunoconjugates thus formed are described in the Examples below.

Various carrier moieties can be employed to produce the immunoconjugates of the present invention. Preferably, the carrier moiety is a protein. For instance, proteins derived from bacteria or viruses, such as tetanus toxoid (TT), diphtheria toxoid or related protein such as diphtheria toxin cross-reactive mutant 197 (CRM), cholera toxoid, members of the LTB family of bacterial toxins, retrovirus nucleoprotein (retro NP), rabies ribonucleoprotein (rabies RNP), vesicular stomatitis virus nucleocapsid protein (VSV-N), recombinant pox virus subunits, and the like may be used. Other suitable carrier moieties include keyhole hemocyanin (KLH), edestin, thyroglobulin, bovine serum albumin (BSA), human serum albumin, red blood cells such as sheep erythrocytes, (SRBC), as well as polyamino acids such as poly(D)lysine, poly(D)glutamic acid and the like. Polymers also can be used, e.g., carbohydrates such as dextran, mannose, or mannan.

There are a wide range of available methods for linking a hapten to a carrier moiety, any of which are suitably adapted for use in the present invention. The linker moiety may be monovalent or divalent depending on whether the carrier moiety is covalently attached to the linker moiety. In some preferred embodiments, the linker moiety X of the heroin haptens contains a simple thiol group. In some other embodiments, the linker moiety comprises an activated acyl. The length and nature of the linker moiety is such that the hapten is displaced a sufficient distance from the carrier moiety to elicit a suitable antibody response to the hapten in vivo. Suitable linker moieties include:
—OY,
—OCH₃,
—OCO(CH₂)ₙCOY,
—OCO(CH₂)ₙCNY,
—OCO(CH₂)ₙY,
—OCOCH═Y,
—OCOCH(O)CH₂,
—OCOCH(OH)CH₂Y,
—OCO(CH₂)ₙCH(OH)CH₂Y,
—OCO(CH₂)ₙCH(O)CH₂Y,
—OCOC₆H₅,
—O(CH₂)ₙY
—CO₂Y,
—COY,
—CO(CH2)ₙCOY,
—CO(CH₂)ₙCNY,
—CONH(CH₂)ₙY,
—CH₂OCO(CH2)ₙCOY,
—CH₂OCO(CH₂)ₙCNY,
—(CH₂)ₙY,
—CH₂Z(CH₂)ₙY,
—(CH₂)n-C₆H₁₀—(CH₂)ₘ—COY
—(CH₂)n-C₆H₄—(CH₂)ₘ—COY
—NH(CH₂)ₙCOY
—NH(CH₂)ₖC₆H₁₀—(CH₂)ₘCOY
—NH(CH₂)ₘC₆H₄—(CH₂)ₚCOY
—NHCO(CH₂)ₙCOY
—NHCO(CH₂)ₖC₆H₁₀—(CH₂)ₘCOY
—NHCO(CH₂)ₘC₆H₄—(CH₂)ₚCOY
—C≡C—(CH₂)ₙNHY
—C≡C—(CH₂)ₙCOY
—C≡C—(CH₂)ₙC₆H₁₀—(CH₂)ₘCOY
—C≡C—(CH₂)ₙC₆H₁₀—(CH₂)ₘNHY
—C≡C—(CH₂)ₘC₆H₄—(CH₂)ₚCOY
—C≡C—(CH₂)ₘC₆H₄—(CH₂)ₚNHY
—CH═CH—(CH₂)ₙNHY
—CH═CH—(CH₂)ₙCOY
—CH═CH—(CH₂)ₙC₆H₁₀—(CH₂)ₘCOY
—CH═CH—(CH₂)ₙC₆H₁₀—(CH₂)ₘNHY
—CH═CH—CH₂)ₘC₆H₄—(CH₂)ₚCOY
—CH═CH—(CH₂)ₘC₆H₄—(CH₂)ₚNHY
—SY
—SCH₂Y
—SCO(CH₂)ₙCOY
—SCH₂(CH₂)ₙY
—(CH₂)ₙ—R₁—(CH₂)ᵣ—R₂—Y
—Z(CH₂)ₙY
—ZCO(CH₂)ₙCOY
   wherein n is an integer from about 0 to about 20, or in some embodiments from about 1 to about 12, from about 2 to about 10, or about 3 to about 6; m is an integer from about 0 to about 6; k is an integer from about 0 to about 20; p is an integer from about 0 to about 6; r is an integer from about 1 to about 20; Z is selected from the group consisting of —O—, —CH₂—, and —NH—; R₁ and R₂ are independently selected from the group consisting of —NHCO—, —CONH—, —CONHNH—, —NHNHCO—, —NHCONH—, —CONHNHCO—, and —S—S—; and Y is selected from the group consisting of —H, —OH, ═CH₂, —CH₃, —OCH₃, —COOH, halogen, acyl, activated acyls, such as 2-nitro-4-sulfobenzoate and N-oxysuccinimidate, alkyl, N-maleimides, imino acylate, isocyanates, isothiocyanates, haloformate, vinylsulfone, imidoester, phenylglyoxalate, hydrazide, alkynyl, azido, amino, N-hydroxysuccinimidate, —O—, —(CH₂)ₘ—, —C(O)—, —S—S—, —NH—, —C(O)O—, —C(O)NH—, —N═N—, —N═N═N—, —CH═CH— and —C≡C—. Other suitable linkers of sufficient length and flexibility may also be used with the present invention. A wide range of reagents and/or active groups may be used to facilitate cross-linking of a hapten to a carrier moiety.

The carrier moiety may be modified by methods known to those skilled in the art to facilitate conjugation to the hapten, e.g., by succinylation. About 1 to about 100 haptens may be conjugated to a carrier moiety, more preferably 1-70, 1-50, 1-25, or 1-10 haptens may coupled to the carrier moiety.

In a related aspect, the invention provides an immunoconjugate of formula (II):

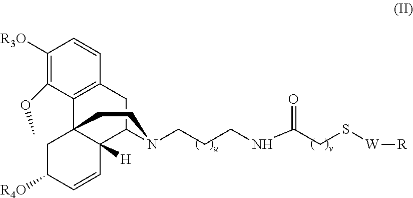

In formula (II), R₃ and R₄ can be any groups that maintain the immunogeneicity of heroin and morphine. In some preferred embodiments, R₃ and R₄ are each —H or an acyl group. u and v in formula (II) are each an integer, e.g., from about 0 to about 6. In formula (II), W is a functional group or linker moiety that facilitates linkage to a carrier moiety, and R is a carrier moiety. In some immunoconjugates of the invention, both R₃ and R₄ are —H. These immunoconjugates may also be termed morphine-like immunoconjugates. In some other immunoconjugates, R₃ and R₄ are both a —COCH₃ group. These compounds may be more specifically termed heroin-like immunoconjugates.

In some preferred embodiments, the linker moiety W in the immunoconjugates of the invention comprises a structure shown in formula (III):

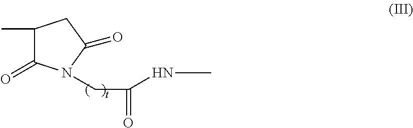

In formula (III), t is an integer, e.g., from about 1 to about 10. In some exemplified immunoconjugates described herein, t is 3 in the linker moiety W. In some other embodiments, the functional group or linker moiety W may be selected from —H, —OH, ═CH₂, —CH₃, —OCH₃, —COOH, halogen, acyl, activated acyls, such as 2-nitro-4-sulfobenzoate and N-oxysuccinimidate, alkyl, N-maleimides, imino acylate, isocyanates, isothiocyanates, haloformate, vinylsulfone, imidoester, phenylglyoxalate, hydrazide, alkynyl, azido, amino, N-hydroxysuccinimidate, —O—, —(CH₂)ₘ— (wherein m is an integer from about 1 to about 20), —C(O)—, —S—S—, —NH—, —C(O)O—, —C(O)NH—, —N═N—, —N═N═N—, —CH═CH— and —C≡C—. Other suitable linkers of sufficient length and flexibility may also be used with the present invention. A wide range of reagents and/or active groups may be used to facilitate cross-linking of a hapten to a carrier moiety. In some preferred embodiments, the linker group W is covalently linked to the carrier moiety R via a covalent bond. In some of these embodiments, the covalent bond is an amide linkage, as exemplified in FIG. 2 herein.

The heroin haptens and immunoconjugates described herein can be produced with standard techniques of organic chemistry. Detailed protocols for synthesizing representative compounds of the invention (e.g., haptens 1-2 and immunoconjugates 11a-12b) are described in the Examples below (e.g., Example 1 and FIG. 2).

### IV. Vaccines Comprising Heroin Immunoconjugates and Their Use for Immunotherapy

The immunoconjugates of the invention can be used to prepare vaccines that are suitable for active immunization protocols. Compositions including the immunoconjugates of the invention can be formulated for in vivo use, e.g., therapeutic or prophylactic administration to a subject. In some particular embodiments, the immunoconjugates are formulated as vaccine compositions.

The preparation of vaccines which contain immunoconjugates as active ingredients is generally well understood in the art. Typically, such vaccines are prepared as injectables, either as liquid solutions or suspensions. Solid forms suitable for formulation in solution or suspension prior to injection may also be prepared. The preparation may also be emulsified. The immunoconjugate may be mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants (e.g., an alum adjuvant) which enhance the effectiveness of the vaccines, as described below. In some pharmaceutical compositions (e.g., vaccines) containing the immunoconjugate of the invention, the intended immune response is enhanced by the inclusion of an adjuvant substance. Adjuvants and their use are well known in the art. Examples of adjuvants include inorganic adjuvants such as aluminum salts (“alums”), e.g., aluminum hydroxide, aluminum phosphate, potassium aluminum sulfate, and aluminum hydroxide and magnesium hydroxide mixture suspension. Suitable adjuvants also include organic adjuvants such as Squalene, oil-based adjuvants, and virosomes which contain a membrane-bound hemagglutinin and neuraminidase derived from the influenza virus. Various methods of achieving an adjuvant effect are also known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9.

Vaccines can be conventionally administered to subjects. Preferably, they are administered parenterally by injection, for example, subcutaneously, intracutaneously, intradermally, subdermally or intramuscularly, or any other routes that are suitable for the present invention. Additional formulations which may be suitable for other modes of administration include suppositories and, in some cases, oral, buccal, sublingual, intraperitoneal, intravaginal, epidural, spinal, and intracranial formulations.

As can be appreciated, compositions of the invention should be administered in a manner compatible with the dosage formulation, and in such amount as will be prophylactically or therapeutically effective and immunogenic. The quantity to be administered depends on the subject to be treated, including, e.g., the capacity of the individual's immune system to mount an immune response, and the degree of protection desired. Suitable dosage ranges are from about 0.1 μg/kg body weight to about 10 mg/kg body weight, such as in the range from about 500 μg/kg body weight to about 1000 μg/kg body weight. For example, a dosage range may be from about 0.1 mg/kg body weight, about 0.25 mg/kg body weight, about 0.5 mg/kg body weight, about 0.75 mg/kg body weight, about 1 mg/kg body weight, or about 2 mg/kg body weight, to about 20 mg/kg body weight, about 15 mg/kg body weight, about 10 mg/kg body weight, about 7.5 mg/kg body weight, or about 5 mg/kg body weight. Suitable regimens for initial administration and booster shots are also contemplated and are typified by an initial administration followed by subsequent inoculations or other administrations.

Some embodiments of the invention provide a method of inducing an anti-heroin immune response in a subject. The subject can be a human or a non-human animal, e.g., a mouse or rat in an animal model. An anti-heroin immune response specifically refers to induction of a therapeutic or prophylactic heroin-sequestering effect that is mediated by the immune system of the subject. Such an immune response suitably promotes clearance or immune control of heroin and its psychoactive metabolites (such as 6AM, M6G, and/or morphine) in the subject. In some embodiments, the anti-heroin immune response is an antibody response. The antibody response may suitably be the production of IgG, IgA, IgM or IgE antibodies. The anti-heroin immune response is suitably assessed by methods known in the art, e.g. ELISA for anti-heroin antibodies. Inducing an anti-heroin immune response in a subject in accordance with the invention may be accomplished by administering to the subject the immunoconjugate compositions described above.

In some embodiments, the methods of the invention are directed to inducing an anti-heroin immune response which provides system-wide effects in the subject. The systemic effects can include, e.g., reduction of heroin withdrawal symptoms. Heroin withdrawal symptoms include, but are not limited to, craving for the drug, restlessness, muscle and bone pain, insomnia, diarrhea, nausea, vomiting, cold flashes, cold sweat, goose bumps, involuntary kicking movements, dilated pupils, watery eyes, runny nose, excessive/repeated yawning, loss of appetite, tremors, panic, muscle cramps, shallow breathing, convulsions, increased heart rate, elevation in pulse, elevated temperature, sharp elevation in blood pressure, arrhythmia, stroke, heart attack, coma, depression and suicidal tendencies.

### V. Antibodies Specific for Heroin Haptens

The present invention also provides antibodies that immunoreact with the haptens (e.g., hapten 1 shown in FIG. 2) of this invention. Unless otherwise specified, the anti-heroin antibodies of the invention encompass antibodies that are specific for one or more of heroin and its psychoactive metabolites (including 6AM, M6G and morphine). Thus, in some embodiments, antibodies of the invention recognize heroin and also cross-react with one or more of the psychoactive metabolites of heroin, e.g., 6AM, morphine and morphine-6-glucuronide (M6G). In some of these embodiments, the antibodies cross-react with 6AM but not morphine. The antibodies may be of any of the immunoglobulin subtypes IgA, IgD, IgG, IgE, or IgM. Antibodies may be produced by any means known in the art and may be, e.g., monoclonal antibodies, polyclonal antibodies, phage display antibodies, and/or human recombinant antibodies. A recombinant antibody can be manipulated or mutated so as to improve its affinity or avidity for the antigen, e.g., 6AM or heroin. Means of such manipulation are well known in the art.

In some embodiments, human antibodies or humanized antibodies may be used in passive immunization protocols. Methods to humanize murine monoclonal antibodies via several techniques may be used and are well known in the art. Further, methodologies for selecting antibodies with desired specificity from combinatorial libraries make human monoclonal antibodies directly available. If desired, protein engineering may be utilized to prepare human IgG constructs for clinical applications such as passive immunization of a subject. In passive immunization, a short-term immunization is achieved by the transfer of antibodies to a subject. The antibodies can be administered in a physiologically acceptable vehicle which can be administered by any suitable route, e.g., intravenous (IV) or intramuscular (IM). Any antibodies of the invention described herein may be suitably used, such as monoclonal antibodies (mAb).

The passive administration of anti-heroin antibodies should prove beneficial to reduce serum levels and attenuate “toxic” (cardiovascular, metabolic, endocrine) effects. It can also be used in weekly or biweekly pharmacotherapy during heroin cessation or opiate cessation programs. The pharmacotherapy could entail self-injection of mAb to maintain a high circulating level of antibody. It may be possible to establish passive mucosal protection against heroin in the respiratory tract through the use of aerosolized immunoglobulin (see, e.g., Crowe et al., Proc. Natl. Acad. Sci. USA 91:1386-1390, 1994).

In some embodiments, active immunization (immunoconjugate vaccine) and passive immunization (antibodies) may be used in combination in a subject. The effective dose of either the immunoconjugate vaccine or antibodies may be the effective dose of either when administered alone. In some embodiments, the effective dose of either in combination with the other may be less than the amount that would be therapeutically effective if either is administered alone.

Some embodiments of the invention provide a method of reducing withdrawal symptoms of heroin in a subject. Reducing withdrawal symptoms of heroin can encompass, but is not limited to, reducing craving for craving for heroine or related drugs, restlessness, muscle and bone pain, insomnia, diarrhea, nausea, vomiting, cold flashes, cold sweat, goose bumps, involuntary kicking movements, dilated pupils, watery eyes, runny nose, excessive/repeated yawning, loss of appetite, tremors, panic, muscle cramps, shallow breathing, convulsions, increased heart rate, elevation in pulse, elevated temperature, sharp elevation in blood pressure, arrhythmia, stroke, heart attack, coma, depression and suicidal tendencies in the subject. Methods of reducing withdrawal symptoms may be accomplished by administering to the subject the immunoconjugate compositions described above in combination with passive immunization or other adjunct therapies used in heroin or opiate cessation.

It will be appreciated that the specific dosage of immunoconjugate or antibodies administered in any given case will be adjusted in accordance with the condition of the subject and other relevant medical factors that may modify the activity of the immunoconjugate or antibody or the response of the subject, as is well known by those skilled in the art. For example, the specific dose for a particular patient depends on age, body weight, general state of health, diet, the timing and mode of administration, the rate of excretion and medicaments used in combination. Dosages for a given patient can be determined using conventional considerations such as by means of an appropriate conventional pharmacological protocol.

It is specifically contemplated that any embodiment of any method or composition of the invention may be used with any other method or composition of the invention. As used in this specification and the appended claims, the singular forms “a,” “an,” and “the” include plural referents unless the content clearly dictates otherwise. Thus, for example, reference to a composition containing “an antibody” includes a mixture of two or more antibodies. It should also be noted that the term “or” is generally employed in its sense including “and/or” unless the content clearly dictates otherwise. It also is specifically understood that any numerical value recited herein includes all values from the lower value to the upper value, i.e., all possible combinations of numerical values between the lowest value and the highest value enumerated are to be considered to be expressly stated in this application. For example, if a range is stated as 1% to 50%, it is intended that values such as 2% to 40%, 10% to 30%, or 1% to 3%, etc., are expressly enumerated in this specification.

The invention is further exemplified by the specific Examples described below.

### EXAMPLES

The following examples are provided to further illustrate the invention but not to limit its scope.

### Example 1

### Experimental Procedures

NMR spectra were recorded on Bruker spectrometers. Chemical shifts are reported in parts per million (ppm). For ¹H NMR spectra (CDCl₃), the residual solvent peak was used as the reference (7.26 ppm), while the central solvent peak was used as the reference (77.0 ppm in CDCl₃) for ¹³C NMR. Preparative HPLC was performed using a Shimadzu LC-8A system equipped with a Grace-Vydac C18 column (2.2 cm×15 cm). All HPLC experiments were monitored at 254 or 214 nm. Analytical thin layer chromatography (TLC) was performed using EMD pre-coated TLC plates, silica gel 60F-254, 0.25 mm layer thickness. TLC plates were visualized by exposure to UV light or submersion in aqueous potassium permanganate followed by heating on a hot plate. Preparative TLC was performed using EMD Silicagel 60F-254 plates (20×20 cm), 0.5 mm thickness. When necessary, reaction vessels were oven dried and cooled in a dessicator and performed under an inert argon atmosphere. Reagents were commercial grade and used without further purification. Heroin hydrochloride was obtained from NIDA, 6-acetyl morphine was prepared according to the procedure described in Neville et al. (Mag. Res. Chem. 1987, 25, 31-35), and Leu-enkephalin/Endomorhin-1 were prepared by standard solid phase peptide synthesis described in Zinieris et al., J. Comb. Chem. 2004, 7:4-6; and Fransson et al., J. Med. Chem. 2010, 53: 2383-2389. Analytical HPLC was used for analysis of compound purity; all purified compounds were of >95% purity, with the exception of Boc-protected compound 6.

Synthesis of (4aR,7S,7aR,12bS)-3-(3-((tert-butoxycarbonyl)amino)propyl)-2,3,4,4a,7,7a-hexahydro-1H-4,12-methanobenzofuro[3,2-e]isoquinoline-7,9-diyl diacetate (6). To a solution of heroin hydrochloride 3 (21 mg/0.052 mmol) in 4 mL of dry 1,2-dichloroethane was added NaHCO₃ (35 mg/0.42 mmol) and α-chloroethylchloroformate (93 μL/0.85 mmol) at room temperature. The resulting solution was heated to reflux for four hours with monitoring by TLC (9:1 CHCl₃:MeOH). After this time, the solution was cooled and filtered before removal of the solvent under reduced pressure. The remaining residue was placed under high vacuum for two hours, followed by the addition of CH₃CN:H₂O (4:1, 0.1% TFA, 2 mL) and stirring for two hours at room temperature. Acetonitrile was removed under reduced pressure, and the aqueous solution lyophilized to give crude norheroin 4 as an amorphous solid (22 mg/90% crude yield). Crude norheroin 4 (22 mg/0.047 mmol) was dissolved in dry CH₂Cl₂ (5 mL) and cooled to 0° C., followed by the addition of aldehyde 5 (25 μL/0.24 mmol), acetic acid (14 μL/0.18 mmol) and NaBH(OAc)₃ (120 mg/0.6 mmol) at the same temperature. The solution was allowed to stir at 0° C. for two hours before allowing to slowly warm to room temperature overnight. After this time, additional aldehyde 5 (25 μL/0.24 mmol) and NaBH(OAc)₃ (60 mg/0.3 mmol) were added and the solution stirred at room temperature for 6 hours before the addition of water (3 mL). The layers were separated, and the aqueous layer extracted with CH₂Cl₂ (3×5 mL). The combined organic layers were washed with brine (1×5 mL), dried with MgSO₄ and the solvent removed under reduced pressure. The resulting residue was purified by preparative TLC (9:1 CHCl₃:MeOH) to give the pure product as an amorphous solid (18 mg/65% yield from 3). ¹H NMR 500 MHz (CDCl₃) δ 6.75 (d, J=8.2 Hz, 1H), 6.56 (d, J=8.2 Hz, 1H), 5.63 (d, J=9.9 Hz, 1H), 5.43 (dt, J=2.5, 10.8 Hz, 1H), 5.24 (br s, 1H), 5.15 (m, 1H), 5.11 (d, J=6.6 Hz, 1H), 3.45 (s, 1H), 3.15 (m, 2H), 2.96 (d, J=18.7 Hz, 1H), 2.77 (m, 1H), 2.67 (m, 1H), 2.54 (m, 2H), 2.36 (m, 1H), 2.32 (m, 1H), 2.27 (s, 3H), 2.12 (s, 3H), 2.08 (m, 1H), 1.87 (m, 1H), 1.55 (m, 4H), 1.44 (s, 9H); ¹³C NMR 125 MHz (CDCl₃) δ 170.6, 168.6, 156.2, 149.5, 132.0, 131.9, 131.5, 129.5, 128.7, 122.2, 119.5, 88.7, 79.2, 68.0, 56.9, 54.3, 44.9, 43.3, 40.4, 40.2, 34.7, 29.3, 28.0, 24.6, 21.7, 20.8, 20.7. High resolution mass spectrometry (ESI) found 527.2761 [calculated for C₂₉H₃₉N₂O₇ (M+H⁺) 527.2752]. Purity (HPLC): 94%.

Synthesis of (4aR,7S,7aR,12bS)-3-(3-(3-tritylthio)propanamido)propyl)-2,3,4,4a,7,7a-hexahydro-1H-4,12-methanobenzofuro[3,2-e]isoquinoline-7,9-diyl diacetate (9). To a solution of Boc-protected amine 6 (18 mg/0.034 mmol) in CH₂Cl₂ (1 mL) at room temperature was added a solution of CH₂Cl₂:TFA (1 mL:1 mL) in one portion. The resulting solution was stirred at room temperature for 2 hours, and then the solvents removed under reduced pressure to give 7 as a yellow solid. This solid was placed under high vacuum overnight, followed by dissolving in CH₂Cl₂ (4 mL) and cooling the resulting solution to 0° C. Triethylamine (17 μL/0.12 mmol) and activated ester 8 (18 mg/0.04 mmol) were added at the same temperature, and the resulting solution was stirred at 0° C. for two hours before allowing to warm to room temperature overnight. The solution was transferred to a separatory funnel, and washed once with brine (1×4 mL). The aqueous layer was extracted with CH₂Cl₂ (2×5 mL), the combined organic layers were dried with MgSO₄, and the solvent removed under reduced pressure to give the crude product as a viscous oil which was purified by preparative TLC (9:1 CHCl₃:MeOH) to give the product as an amorphous solid (18 mg/71% yield from 6). ¹H NMR 500 MHz (CDCl₃) δ 7.55-7.25 (m, 15H), 6.85 (d, J=8.2 Hz, 1H), 6.65 (d, J=8.2 Hz, 1H), 5.70 (m, 2H), 5.45 (dt, J=2.4, 10.0 Hz, 1H), 5.23 (m, 1H), 5.16 (d, J=6.6 Hz, 1H), 3.52 (m, 1H), 3.30 (m, 2H), 3.04 (d, J=18.8 Hz, 1H), 2.81 (m, 1H), 2.72 (m, 1H), 2.63 (m, 2H), 2.57 (t, J=7.2 Hz, 2H), 2.43 (m, 1H), 2.37 (m, 1H), 2.36 (2, 3H), 2.22 (s, 3H), 2.12 (t, J=7.1 Hz, 2H), 2.05 (m, 1H), 1.90 (m, 1H), 1.65 (m, 4H); ¹³C NMR 125 MHz (CDCl₃) δ 171.1, 170.6, 168.6, 149.4, 144.7, 132.0, 131.9, 131.6, 129.7, 129.5, 128.6,128.0 126.8, 122.0, 119.5, 88.7, 68.0, 66.8, 57.0, 54.3, 44.8, 43.4, 40.5, 39.3, 35.9, 35.1, 27.9, 27.5, 24.8, 21.8, 20.7, 20.6. High resolution mass spectrometry (ESI) found 757.3326 [calculated for C₄₆H₄₉N₂O₆S (M+H⁺)757.3306].

Synthesis of N-(3-((4aR,7S,7aR,12bS)-7,9-dihydroxy-4,4a,7,7a-tetrahydro-1H-4,12-methanobenzofuro[3,2-e]isoquinolin-3(2H)-yl)propyl-3-(tritylthio)propanamide (10). To a solution of trityl protected heroin hapten 9 (12 mg/0.15 mmol) in MeOH (2 mL) was added 0.1 M NaOH (1 mL) at room temperature. The resulting solution was stirred at room temperature for 45 minutes before the removal of MeOH under reduced pressure. The remaining aqueous phase was extracted with EtOAc (6×5 mL), the combined organics were dried with MgSO₄ and the solvent removed under reduced pressure to give the crude product as a viscous oil which was purified by preparative TLC (9:1 CHCl₃:MeOH) to give the pure product as an amorphous solid (9.5 mg/92% yield). ¹H NMR 600 MHz (CDCL₃) δ 7.40-7.20 (m, 15H), 6.70 (d, J=8.1 Hz, 1H), 6.58 (d, J=8.1 Hz, 1H), 5.78 (br s, 1H), 5.75 (d, J=11.0 Hz, 1H), 5.29 (d, J=9.7 Hz, 1H), 4.96 (d, J=5.9 Hz, 1H), 4.25 (m, 1H), 3.57 (m, 1H), 3.28 (m, 2H), 3.0 (d, J=18.5 Hz, 1H), 2.69 (m, 1H), 2.54 (m, 2H), 2.50 (t, J=7.3 Hz, 2H), 2.40 (m, 1H), 2.30-2.45 (m, 5H), 2.15 (t, J=7.1 Hz, 2H), 1.90 (m, 1H), 1.70 (m, 4H); ¹³C NMR 150 MHz (CDCl₃) δ 170.2, 145.3, 144.8, 138.1, 133.1, 130.9, 129.7, 128.1, 128.0, 126.9, 121.9, 120.1, 116.8, 91.8, 66.9, 66.6, 56.7, 54.3, 44.9, 43.8, 40.7, 39.4, 35.9, 30.9, 27.9, 27.4, 22.8, 21.4. High resolution mass spectrometry (ESI) found 673.3100 [calculated for C₄₂H₄₅N₂O₄S (M+H⁺) 673.3094].

Synthesis of (4aR,7S,7aR,12bS)-3-(3-(3-mercaptopropanamido)propyl-2,3,4,4a,7a-hexahydro-1H-4,12-methanobenzofuro[3,2-e]isoquinoline-7,9-diyl diacetate (1). 5 mg of trityl protected hapten 9 was placed in a round bottom flask and put under high vacuum overnight, followed by purging the flask with Argon. CH₂Cl₂ (1 mL) was then added to the flask, followed by addition of a solution of CH₂Cl₂:TIPS:TFA (1 mL:33 μL:33 μL) at once at room temperature. The resulting solution was purged briefly with Argon, and stirred at room temperature for two hours before removal of the solvent under vacuum. The crude product was purified by preparative HPLC (0 to 10 min at 10% B, 10 to 50 min gradient to 90% B, 50 to 55 min gradient to 10% B, 55 to 60 min at 10% B) to yield fractions containing the pure thiol 1. CH₃CN and TFA were removed under reduced pressure followed by lyophilization of the remaining aqueous solution to give the pure thiol as a white powder (≈2 mg). This thiol was sensitive to oxidation to disulfide, and was used immediately for protein conjugation.

Synthesis of N-(3-((4aR,7S,7aR,12bS)-7,9-dihydroxy-4,4a,7,7a-tetrahydro-1H-4,12-methanobenzofuro[3,2-e]isoquinolin-3(2H)-yl)-3-mercaptopropanamide (2). Synthesized using a procedure analogous to that for the synthesis of 1.

Construction of Heroin and Morphine Immunoconjugates 11a-12b. To a 0.1 mM solution of KLH or BSA (Pierce Protein Research Products) was rapidly added 10-fold excess of a solution of Sulfo-GMBS (N-[g-Maleimidobutyryloxy]sulfosuccinimide ester, Pierce Protein Research Products, 10 mM stock concentration, 1 mM final concentration) at room temperature. The resulting solution was gently shaken at room temperature for 30 minutes, followed by the removal of excess Sulfo-GMBS by dialysis (Slide-A-Lyzer cassette, 10,000 MWCO, Pierce, PBS buffer, pH=7.4) at 4° C. (we found the use of ‘preactivated’ KLH/BSA (Pierce) yielded significant amounts of protein denaturation during hapten conjugation, thus BSA/KLH was always ‘activated’ following this standard procedure). To the solution of ‘activated’ KLH obtained after dialysis was added heroin or morphine hapten 1-2 (2 mg hapten in 350 μL PBS+30 μL DMSO, 1 mg hapten: 1 mg of protein) at 4° C., and the resulting solution gently shaken at the same temperature for four hours. After this time-period, the crude immunoconjugate was purified by dialysis (PBS, pH=7.0) at 4° C. to give pure immunoconjugate in 0.5-1.0 mg/mL concentration as measured by bicinchoninic acid (BCA) assay (Pierce). Coupling efficiency for BSA immunoconjugates 1-2 was monitored by MALDI-TOF MS, and found to be ≈22 copies of hapten per protein. It was assumed that coupling efficiency for KLH protein conjugates was similar, as KLH can not be analyzed by MALDI-TOF.

Active Immunization Protocol. All procedures adhered to the National Institutes of Health Guide for the Care and Use of Laboratory Animals and were approved by the Institutional Animal Care and Use Committee of The Scripps Research Institute. Four groups of n=8 male wistar rats (n=24 total; Charles River, Raleigh, N.C.) weighing between 225-275 g at the beginning of immunization were used. All rats were housed in groups of 3 per cage in a temperature-controlled (22° C.) vivarium on a 12 h light/dark cycle (lights on at 6:00 h) with ad libitum access to food and water. Rats were assigned to morphine (MOR), heroin (HER) or control (KLH) vaccine groups. Rats were immunized with 0.1 mg of immunoconjugate 11b (HER) or 12b (MOR) in formulation with Alum adjuvant (Imject, Pierce) administered into 3 sites (2 s.c. and 1 i.p.). Six total immunizations were performed during the course of the study at days 0, 14, 28, 53, 108 and 151. On days 14, 28, 53, 75, 105, 116 and 130 roughly 0.2 mL of serum was collected to determine immune response as measured by ELISA. At day 165 the rats were sacrificed and their blood collected. While our protocol was not designed to specifically address vaccine toxicity, we did not notice any obvious signs of inflammation or irritation on subsequent booster injection sites. Weights of vaccinated animals were healthy and had no signs of chronic health issues.

ELISA and Competition ELISA. ELISAs to determine limiting dilution were performed using Costar 3690½ area high binding affinity plates. Plates were coated with BSA conjugated heroin or BSA conjugated morphine at 0.5 μg/mL in pH 6.4 PBS and dried overnight at 37° C. Phosphate buffered saline of pH 6.4 was used throughout the ELISAs due to hydrolysis of the 3′-acetyl group of heroin in neutral to basic pHs at elevated temperature. Rat sera samples were diluted in 2% BSA serially across the plate to a final concentration of 1:4,096,000. Plates were developed using a goat α rat HRP secondary antibody as well as TMB substrate (Pierce, Rockford, Ill.). Absorbance was read on a Spectramax M2^{e} 250 spectrophotometer with endpoint readings at 450 nm. O.D. values were plotted on a non-linear curve algorithm using PRISM software.

Competition ELISAs were performed using rat sera samples from bleed 3, due to the highest recorded antibody titers throughout the 165 day experiment. Sera samples were diluted to O.D.₅₀ titer levels determined previously. 10 mM drug stocks were prepared in DMSO. Stocks were diluted directly into coated 96 well costar 3690 plates to a final concentration range of 100 μM-49 nM (<1% DMSO per well). Plates were developed as described previously. Absorbance was read at 450 nm and O.D. values were plotted in PRISM in a one site fit log IC₅₀ non-linear curve model to determine inhibition constants of drug competitors.

Radioimmunoassay (Equilibrium Dialysis). Equilibrium dialysis was performed using Harvard Apparatus 96-well equilibrium dialyzer plates, MWCO 5 kDa. All equilibrium dialysis was conducted at pH 7.0. To each side of the plate was added ˜15,000 dpm of ³H Morphine (American Radiolabeled Chemicals, 80 Ci/mmol, 1 mCi/mL) in 25 μL, 2% BSA. On one side of the plate was added 75 μL of PBS buffer and 50 μL heroin/morphine of the appropriate dilution. On the other side of the plate was added 75 μL of diluted sera (due to supply constraints, sera from the 8^{th} bleed was used for all equilibrium dialysis experiments) in 2% BSA and 50 μL of heroin/morphine of the appropriate dilution. The plate was then rotated for 22 hours, followed by removal of 75 μL it from each side of the plate, and dissolution into 5 mL of scintiallation fluid (Ecolite, MP Biomedicals). Counts of radioactivity, as measured in dpm, were calculated (Beckman LS6500 Liquid Scintillation Counter). Percent inhibition was determined using the method of Muller (Meth. Enzymol. 1983, 92:589-601), with IC50 values determined using Prism software. IgG was assumed to have a molecular weight of 150 kDa and two binding sites per molecule.

Determination of Vaccine's Ability to Blunt the Antinociceptive Effects of Heroin. Behavior experiments began after the 4^{th} vaccine injection (3^{rd} boost), when antibody titers were elevated. For the hot plate test (Ugo Basile, model-DS37), a steady temperature of 54±0.2° C. was used to evaluate thermal nociception. The animals were placed in an acrylic cylinder of 24-cm diameter on the heated metal surface, and the time between placement and hind paw licking or jumping (whichever occurred first) was recorded as nociceptive latency. A 45-s cut-off was established to prevent tissue damage. Rats were tested on the hot plate before (0; baseline) and 30 min after heroin (1 mg/kg; s.c.) or oxycodone (2.5 mg/kg; s.c.) injections. Mechanical nociceptive thresholds (von Frey's test) were measured according to King's method (King et al., Nat. Neurosci. 2009, 12:1364-1366). Briefly, rats were acclimated for 30 minutes in elevated acrylic cages with a wire mesh floor. A series of von Frey filaments were applied perpendicularly to the plantar surface of the hindpaw for 3 seconds. A sharp withdrawal of the hindpaw indicated a positive response. The stimulus was incrementally increased until a positive response was obtained, then decreased until a negative result was obtained in order to determine a pattern of responses for analysis by the non-parametric method of Dixon (Dixon et al., Ann. Rev. Pharmacol. Toxicol. 1980, 20:441-462).

Self Administration Studies. For heroin self-administration experiments, rats prepared with chronic intravenous Silastic catheters (Dow Corning, USA) into the right jugular vein and tested for self-administration in standard operant chambers (Med Associates Inc., St. Albans, Vt.) as described in Vendruscolo et al., Pharmacol. Biochem. Behay. 2011, 98:570-4. The rats had to press one of the two levers (the active lever) on a fixed-ratio (FR) 1 schedule (each response resulted in fluid delivery) to obtain 0.1 ml (over 4 s) of heroin (60 μg/kg/infusion) in 1 h sessions. Reinforced responses were followed by a 20 s time-out period, in which a cue-light (above the active lever) was turned on and lever presses did not result in additional injections. Presses on the other lever (inactive lever) had no programmed consequences. Rats were tested for 14 sessions and the criterion for acquisition of heroin self-administration was a minimum of 3 reinforcements per session over 3 consecutive sessions. Rats were also tested for self-administration of a very palatable sweet solution in order to verify whether differences in acquisition of heroin self-administration among groups were specific for heroin or generalized to other reinforcers (i.e., differences in learning an operant task). For this experiment, animals were tested in similar operant chambers as for heroin, but a drinking cup placed at equidistance of the levers 6 cm from the floor was present for fluid delivery. In a 30 min session, presses in the active lever resulted in the delivery of 0.1 ml of a sweet solution (3% glucose+0.125% saccharin in water). The active lever (left side) was opposite in relation to the active lever used for heroin self-administration (right side) in order to control for acquisition differences during the heroin experiment, i.e., the position of the active lever was novel for all the animals. Criteria for the acquisition of sweet solution intake were defined as 30 or more responses on the active lever in consecutive sessions.

Statistical Analysis. The results are presented as means and standard error of the mean (SEM). Hot plate and von Frey data were analyzed using analysis of variance (ANOVA) for repeated measures with group (KLH, HER and MOR) as a between-subject factor and time (baseline and post-injection) as a within-subject factor. The Newman-Keuls test was used for post-hoc comparisons of the means when appropriate. To analyze acquisition rates, a Kaplan-Meier survival analysis was performed on the number of sessions required to reach criterion followed by a logrank test (GraphPad Prism version 4.03, GraphPad Software, USA). The accepted level of significance for all tests was p<0.05.

### Example 2

### Synthesis of Haptens and Immunoconjugates

This Example describes synthesis of heroin-like and morphine-like haptens 1-2, and their conjugation to carrier Proteins. Synthesis of haptens 1-2 commenced from heroin hydrochloride salt 3, which was demethylated using a modification of Olofson's procedure (Olofson et al., Pure Appl. Chem. 1988, 60, 1715-1724.). Thus, 3 was heated with α-chloroethyl chloroformate (ACE-Cl) and NaHCO₃ to form the requisite intermediate carbamate. Following filtration to remove NaHCO₃, the crude carbamate was decomposed in 6:1 CH₃CN:H₂O containing 0.1% trifluoroacetic acid (TFA) at room temperature to give norheroin 4 in 90% crude yield. This modification gave reliable yields of norheroin 4 without decomposition of heroin's 3′phenolic ester, which commonly occurred when the carbamate was decomposed by the original procedure of warming in MeOH. Reductive amination between N-Boc-δ-aminobutanal 5 (Boeglin et al., J. Comb. Chem. 2005, 7, 864-878) and crude norheroin 4 using NaBH(OAc)₃ gave Boc-protected amine 6 in 65% yield from heroin hydrochloride 3. Acidic deprotection of 6 yielded primary amine 7, which was coupled without purification with trityl-protected thiol 8 (Aust. J. Chem. 1990, 43:629-634) to give trityl protected heroin hapten 9 in 70% yield from Boc-protected amine 6. At this point, trityl protected morphine hapten 10 was synthesized in 63% yield from Boc-protected amine 6 by saponification of protected heroin hapten 9. The trityl protecting group of heroin/morphine haptens 9-10 was then removed under acidic conditions to give thiols 1-2, followed by preparative HPLC purification and conjugation to maleimide activated keyhole limpet hemocyanin (KLH) or BSA to give immunoconjugates 11a-12b (see the synthesis scheme shown in FIG. 2).

### Example 3

### Immunogenicity of Heroin Vaccine in Rats

To assess vaccine immunogenicity, three groups of male. Wistar rats (n=8 per group) were vaccinated with heroin-like immunoconjugate-11b, morphine-like immunoconjugate-12b, or KLH carrier protein (negative control). Rats were immunized with immunoconjugate in formulation with Alum adjuvant. Following a vaccination procedure developed in our laboratories to give optimized titer levels, six total immunizations were performed during the course of the study at days 0, 14, 28, 53, 108 and 151. Rats were bled immediately prior to boosting (t=14, 28, 53 days), and at other regular intervals (t=74, 105, 116, 130, 165 days), to monitor antibody titer levels, with serum obtained after the first injection (t=14 days) showing significant amounts of antibody production as measured by ELISA. We observed a steady increase in titer levels for both vaccines, with maximum titer levels occurring after the third injection (second boost, t=53 days). Titers were not observed for the KLH negative control. In comparing the vaccines, maximum antibody titer levels achieved after three injections (second boost) were highest with respect to the morphine-like vaccine (≈1:160,000), while slightly lower with the heroin-like vaccine (≈1:122,000). The difference between high and low responders for the heroin- and morphine-like vaccines at peak titer levels was ≈4-5 fold.

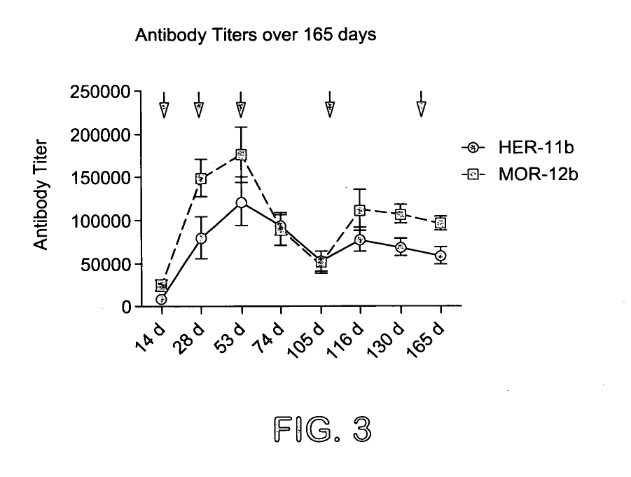

In order to assess antibody decay rate, titer levels were measured at 21 and 53 days (t=74, 105 days after t=0 days) after the fourth injection (third boost). As expected, after this time period antibody titer levels had decreased, but were still respectable for both vaccines (≈1:50,000 at t=53 days). After interruption of the vaccination schedule to determine antibody decay rates, the rats were again immunized (fifth injection/fourth boost) with an increase in titer levels observed after 8 days, showing the vaccine's ability to rapidly regenerate titer levels even after a period of immunization absence (FIG. 3).

### Example 4

### Determination of Anti-Heroin Antibody Affinity

While high titer levels are a critical component for the construction of a successful vaccine, an additional facet that must be considered is the ability of the polyclonal antibodies generated to bind their target with high fidelity. To determine antibody specificity for heroin, 6AM and morphine, competition ELISA was utilized. Thus, antisera from rats vaccinated with heroin-like immunoconjugate-11b bound both heroin and 6AM with high affinity while morphine was bound with decreased affinity. Antisera from rats vaccinated with morphine-like immunoconjugate-12b bound morphine with high affinity, heroin with decreased affinity and did not bind 6AM (Table 1). For all vaccine groups there was no significant cross-reactivity with morphine-6-glucuronide, opioid peptides endomorphin-2/Leu-enkephalin, codeine, opioid agonist methadone and the opioid antagonists naltrexone and naloxone. The importance of the latter finding is that these vaccines could be used in combination with other heroin rehabilitation therapies.

TABLE 1
Competition ELISA data obtained from immunoconjugates 11b and 12b¹.

| | Heroin K_{d} | 6AM K_{d} | Morphine K_{d} |
|---|---|---|---|
| Conjugate | (μM)² | (μM) | (μM) |
| 11b | 4.19 ± 1.01 | 0.0356 ± 0.0010 | 11.2 ± 1.11 |
| 12b | 14.18 ± 6.62 | >100 | 1.18 ± 0.19 |

¹Data represented is the pooled mean sera value ± SEM.
²Sera from the third bleed was used for all competition ELISA experiments.

In order to obtain binding constants for morphine antibody-antigen interactions via an alternative method, a radioimmunoassay (RIA) (Muller, Meth. Enzymol. 1983, 92, 589-601) was performed using ³H morphine. From the RIA, morphine binding affinities for antibodies from immunoconjugates 11b and 12b were 24.5±0.8 nM and 16.6±4.9 nM, respectively. Morphine binding capacity for 11b and 12b calculated from this data was 1.05±0.03 and 9.48±2.81 μM, respectively, corresponding to morphine specific antibody of 0.31±0.01 and 2.84±0.84 mg/mL. A limitation of this technique lies in the fact that ³H heroin and ³H 6AM are not readily available, preventing determination of binding affinity for heroin and 6AM by this method. Consequently, competition ELISA data was used as the guideline for antibody affinity to heroin, 6AM and morphine.

### Example 5

### Ability of Heroin Vaccines to Block Heroin Antinociceptive Effect in Rats

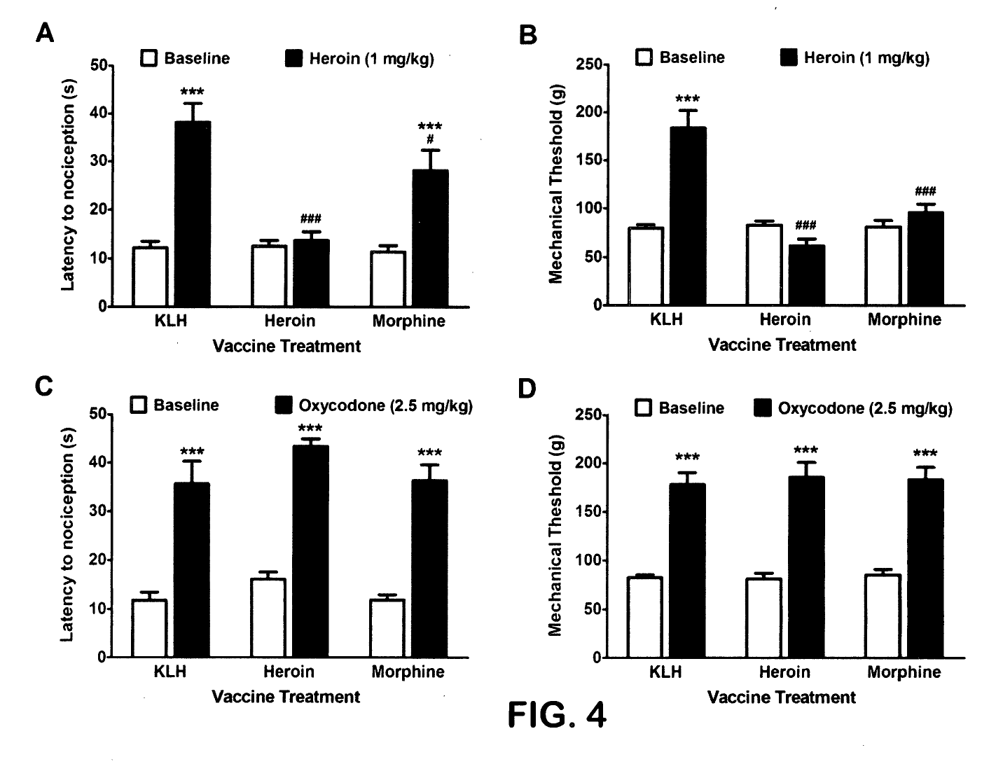

To test the robustness of the vaccine's immune response, mechanical and thermal nociceptive responses were measured following subcutaneous (s.c.) heroin administration using von Frey filaments and the hot plate test, respectively. Thus, after rats had received their fourth course of inoculations (third boost), all groups were administered an established dose of heroin (1 mg/kg, s.c.) that produced pronounced antinociceptive effects. KLH control rats showed marked increases in latency to demonstrate nociceptive behavior on the hot plate (FIG. 4A). Heroin administration also resulted in KLH controls requiring increased force applied to the hindpaw before withdrawal response compared with baseline levels (p<0.001). The rats vaccinated with heroin-like immunoconjugate-11b showed no significant difference from baseline response in either the thermal or mechanical sensitivity tests. The morphine-like immunoconjugate-12b vaccinated rats showed a significant thermal antinociceptive response to heroin (p<0.001), though partially blunted compared to KLH controls (p<0.05). Conversely, morphine vaccine rats did not show a heroin-induced antinociceptive effect in the von Frey test compared to baseline (FIG. 4B).

As a measure of vaccine drug specificity, nociceptive responses to oxycodone in vaccinated rats was tested. Oxycodone is a commonly prescribed analgesic drug that is structurally similar to both heroin and morphine, and provides an excellent challenge for hapten-antibody fidelity. Thus, an analgesic dose of oxycodone (2.5 mg/kg, s.c.) produced near-full thermal antinociceptive effects at 30 min (FIG. 4C) regardless of vaccine pretreatment (p<0.001). A similar pattern of findings was seen in mechanical sensitivity (FIG. 4D), as all the vaccinated groups responded to the oxycodone with significantly increased thresholds compared with baseline levels (p<0.001).

### Example 6

### Ability of Heroin Vaccines to Block Heroin Self-Administration by Rats

Having confirmed the ability of our vaccines to blunt the antinociceptive effects of heroin, the ability of each vaccine to prevent acquisition of heroin self-administration in rats was assessed. Self-administration is the most accepted model of drug addiction, and rats will readily self-administer heroin intravenously without intervention, which can lead to drug dependence (see, e.g., Walker et al., Pharmacol. Biochem. Behay. 2003, 75: 349-354). Accordingly, any vaccine candidate(s) preventing acquisition of heroin self administration in rats could be an attractive possibility for transfer to human trials.

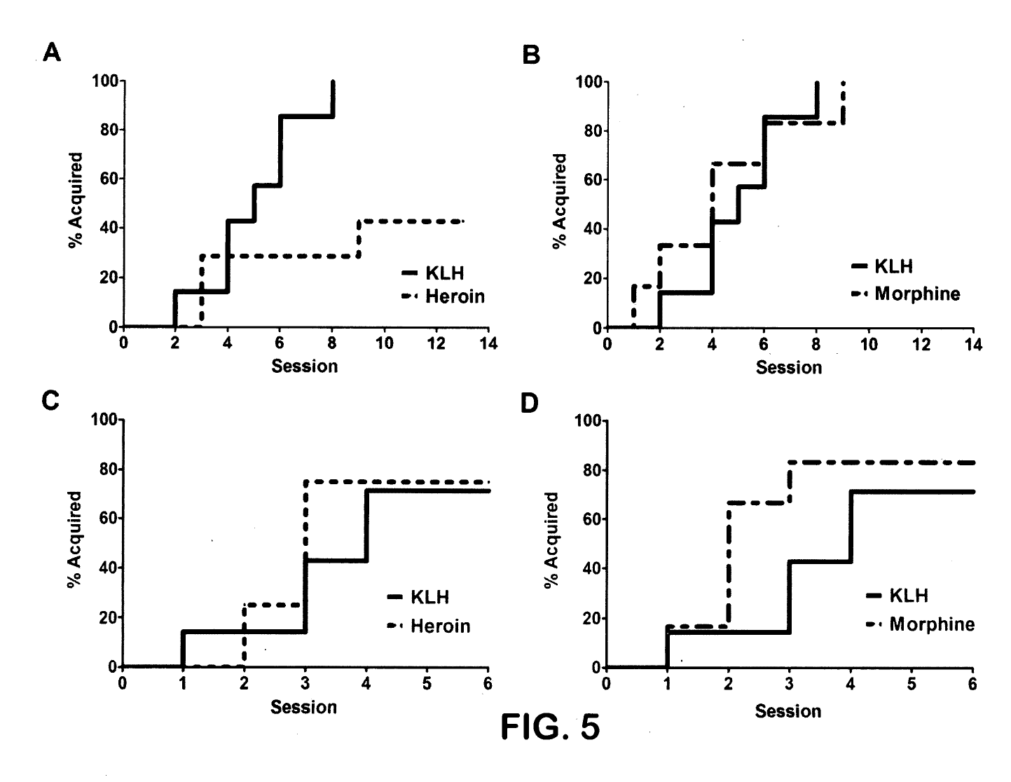

To conduct an acquisition study rats were given a 5^{th} injection (4^{th} boost) of their respective vaccine just prior to surgery to implant intravenous catheters. After a week of recovery, all rats were allowed one hour of access to heroin in the operant chambers. Presses on the active lever were monitored, and most rats pressed the lever at least once in the first session. Those that did not press on their own within the first 20 min during the first 3-4 sessions were given priming injections by guiding the animals to the active lever and depressing it directly in front of them. Those animals that did not press the active lever in subsequent sessions had a short wooden tongue depressor attached to the lever to promote pressing for the 5^{th} and 7^{th} sessions. As seen in FIGS. 5A-5B, all KLH control animals began learning to press the active lever for heroin infusions within a week, defined as pressing three or more times for three consecutive sessions. Rats vaccinated with morphine-like immunoconjugate-12b showed similar ability to controls to acquire heroin self-administration. However, the majority of heroin-like immunoconjugate-11b vaccinated rats failed to maintain pressing for heroin (FIG. 5A). A survival analysis on which animals acquired heroin self-administration showed the ‘heroin’ vaccine significantly reduced the likelihood of acquisition [χ²=5.0, df=1, p<0.05].

### Example 7

### Heroin Vaccines are Specific for Heroin

This Example describes studies which show neither opioid vaccine alters acquisition of self-administration of a natural reward. After a brief period without further testing or training, we sought to validate that the heroin-vaccinated rats were still capable of acquiring lever-pressing behavior to a highly palatable substance (sweetened water). As seen in FIGS. 5C-5D, the majority of the rats from each of the vaccination groups showed similar and rapid acquisition of lever responding for this palatable solution, resulting in hundreds of responses in the 30 min period within less than five sessions. A few rats from each vaccine group showed minimal responding on the active lever (<10 responses per session) and were not likely to meet criteria. However, it should be noted that all animals that failed to acquire responding for the sweetened water did meet criteria in the heroin acquisition. Also, due to the rapid acquisition by most subjects, no priming or additional prompting was necessary for acquisition in this test.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, patents, and patent applications cited in this specification are herein incorporated by reference as if each was specifically and individually indicated to be incorporated by reference.

What is claimed is:

1. A hapten of formula (I): 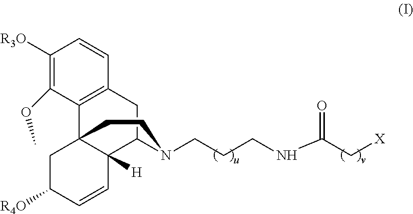
wherein R₃ and R₄ are each —H or an acyl group, u and v are each an integer from about 0 to about 6, and X is a linker moiety.

2. The hapten of claim 1, wherein X is selected from the group consisting of:
—Y,
—CH₃,
—(CH₂)ₙCOY,
—(CH₂)ₙCNY,
—(CH₂)ₙY,
—CH═Y,
—CH(O)CH₂,
—CH(OH)CH₂Y,
—(CH₂)ₙCH(OH)CH₂Y,
—(CH₂)ₙCH(O)CH₂Y,
—C₆H₅,
—(CH₂)ₙY
—NH(CH₂)ₙY,
—CH₂OCO(CH2)ₙCOY,
—CH₂OCO(CH₂)ₙCNY,
—CH₂Z(CH₂)ₙY,
—(CH₂)n-C₆H₁₀—(CH₂)ₘ—COY,
—(CH₂)n-C₆H₄—(CH₂)ₘ—COY,
—NH(CH₂)ₙCOY,
—NH(CH₂)ₖC₆H₁₀—(CH₂)ₘCOY,
—NH(CH₂)ₘC₆H₄—(CH₂)ₚCOY,
—NHCO(CH₂)ₙCOY,
—NHCO(CH₂)ₖC₆H₁₀—(CH₂)ₘCOY,
—NHCO(CH₂)ₘC₆H₄—(CH₂)ₚCOY,
—C≡C—(CH₂)ₙNHY,
—C≡C—(CH₂)ₙCOY,
—C≡C—(CH₂)ₙC₆H₁₀—(CH₂)ₘCOY,
—C≡C—(CH₂)ₙC₆H₁₀—(CH₂)ₘNHY,
—C≡C—(CH₂)ₘC₆H₄—(CH₂)ₚCOY,
—C≡C—(CH₂)ₘC₆H₄—(CH₂)ₚNHY,
—CH═CH—(CH₂)ₙNHY,
—CH═CH—(CH₂)ₙCOY,
—CH═CH—(CH₂)ₙC₆H₁₀—(CH₂)ₘCOY,
—CH═CH—(CH₂)ₙC₆H₁₀—(CH₂)ₘNHY,
—CH═CH—CH₂)ₘC₆H₄—(CH₂)ₚCOY,
—CH═CH—(CH₂)ₘC₆H₄—(CH₂)ₚNHY,
—(CH₂)ₙ—R3-(CH₂)ᵣ—R₂—Y,
—Z(CH₂)ₙY,
—ZCO(CH₂)ₙCOY
wherein n is an integer from about 1 to about 20; m is an integer from about 0 to about 6; k is an integer from about 0 to about 20; p is an integer from about 0 to about 6; r is an integer from about 1 to about 20; Z is selected from the group consisting of —O—, —CH₂—, and —NH—; R₁ and R₂ are independently selected from the group consisting of —NHCO—, —CONH—, —CONHNH—, —NHNHCO—, —NHCONH—, —CONHNHCO—, and —S—S—; and Y is selected from the group consisting of —SH, —H, —OH, ═CH₂, —CH₃, —OCH₃, —COOH, halogen, acyl, 2-nitro-4-sulfobenzoate, N-oxysuccinimididate, N-maleimides, imino acylate, isocyanates, isothiocyanates, haloformate, vinylsulfone, imidoester, phenylglyoxalate, hydrazide, azido, amino, and N-hydroxysuccinimidate.

3. The hapten of claim 1, wherein R₃ and R₄ are each —H.

4. The hapten of claim 1, wherein R₃ and R₄ are each —COCH₃.

5. The hapten of claim 1, wherein u is 2.

6. The hapten of claim 1, wherein v is 2.

7. The hapten of claim 1, wherein X is —SH.

8. The hapten of claim 3, wherein u and v are each 2, and X is —SH.

9. The hapten of claim 4, wherein u and v are each 2, and X is —SH.

10. An immunoconjugate of formula (II): 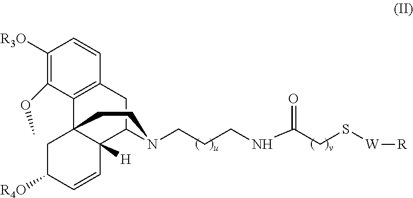
wherein R₃ and R₄ are each —H or an acyl group, u and v are each an integer from about 0 to about 6, and W is a linker moiety that is covalently linked to a carrier moiety R.

11. The immunoconjugate of claim 10, wherein W is a linker moiety of formula (III): 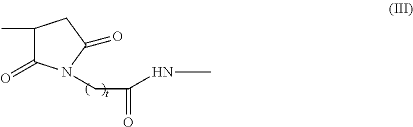
wherein t is an integer from about 1 to about 10.

12. The immunoconjugate of claim 10, wherein R₃ and R₄ are each —H.

13. The immunoconjugate of claim 10, wherein R₃ and R₄ are each —COCH₃.

14. The immunoconjugate of claim 10, wherein R is selected from the group comprising keyhole limpet hemocyanin (KLH), edestin, thyroglobulin, human serum albumin, sheep red blood cells (sheep erythrocytes), tetanus toxoid (TT), diphtheria toxoid, cholera toxoid, polyamino acids, D-lysine, D-glutamic acid, members of the LTB family of bacterial toxins, retrovirus nucleoprotein (retro NP), rabies ribonucleoprotein (rabies RNP), vesicular stomatitis virus nucleocapsid protein (VSV-N), recombinant pox virus subunits, and bovine serum albumin (BSA).

15. The immunoconjugate of claim 10, wherein the carrier moiety is KLH or BSA.

16. The immunoconjugate of claim 11, wherein R₃ and R₄ are each —H, u and v are each 2, t is 3, and the carrier moiety is KLH or BSA.

17. The immunoconjugate of claim 11, wherein R₃ and R₄ are each —COCH₃, u and v are each 2, t is 3, and the carrier moiety is KLH or BSA.

18. A composition comprising an immunologically effective amount of the immunoconjugate of claim 10 and a physiologically acceptable vehicle.

19. The composition of claim 18, further comprising an adjuvant.

20. A method of inducing an anti-heroin immune response in a subject comprising immunizing the subject with an immunologically effective amount of the composition of claim 18.

21. The method of claim 20, wherein R₃ and R₄ are each —COCH₃, u and v are each 2, the carrier moiety is KLH, and W is

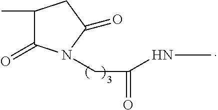

22. An antibody that binds the immunoconjugate of claim 10.

23. The antibody of claim 22, wherein the antibody specifically binds heroin, morphine, acetylmorphine (6AM) or morphine-6-Glucuronide (M6G).

24. The antibody of claim 22, wherein the antibody binds heroin or acetylmorphine (6AM) with a dissociation constant of about 10 nM to about 20 μM.
